(19) United States
(12) Patent Application Publication
(10) Pub. No.: US 2004/0220253 A1
(43) Pub. Date: Nov. 4, 2004

(54) Synthesis of pyrrole amides

(75) Inventor: John A. Ragan, Gales Ferry, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/859,855

(22) Filed: Jun. 3, 2004

Related U.S. Application Data
(62) Division of application No. 10/008,294, filed on Dec. 3, 2001, now abandoned.
(60) Provisional application No. 60/250,666, filed on Dec. 4, 2000.

(51) Int. Cl. C07D 209/42

(52) U.S. Cl. 514/419; 548/492

(57) ABSTRACT

A method for preparing particular pyrrole-carboxamides which selectively bind to GABAa receptors; which comprises reacting 1,3-cycloalkanediones with bromoethylacetate followed by reaction of the resulting product with an acid halide followed by reaction with an aromatic amine and finally with an amonium source at an elevated temperature.

### BACKGROUND OF THE INVENTION

1. Field of the Invention

This invention relates a method for synthesis of a group of fused pyrrolecarboxamides. The compounds selectively bind to GABAa receptors. This invention also relates to chemical intermediates for synthesis of such compounds. Compounds which bind to GABAa receptors are useful in treating anxiety, sleep and seizure disorders, and overdoses of benzodiazepine-type drugs, and enhancing alertness.

2. Description of the Related Art

γ-Aminobutyric acid (GABA) is regarded as one of the major inhibitory amino acid transmitters in the mammalian brain. Over 30 years have elapsed since its presence in the brain was demonstrated (Roberts & Frankel, J. Biol. Chem 187: 55-63, 1950; Udenfriend, J. Biol. Chem. 187: 65-69, 1950). Since that time, an enormous amount of effort has been devoted to implicating GABA in the etiology of seizure disorders, sleep, anxiety and cognition (Tallman and Gallager, Ann. Rev. Neuroscience 8: 21-44, 1985).

1,4-Benzodiazepines continue to be among the most widely used drugs in the world. Principal among the benzodiazepines marketed are chlordiazepoxide, diazepam, flurazepam, and triazolam. These compounds are widely used as anxiolytics, sedative-hypnotics, muscle relaxants, and anticonvulsants.

Certain fused pyrrolecarboxamides which are useful as GABA brain receptor ligands are disclosed in U.S. Pat. No. 5,484,944.

A method for producing pyrrole amides is described in WO 97/26243 which involves protection of the nitrogen in the pyrrole ring. Compounds of PCT/US00/23862 are described as produced by this method.

A method for producing certain pyrrole amides is described in WO 99/25684 that employs a furan-carboxamide intermediate, and avoids protecting the pyrrole nitrogen and carboxylic acid groups as in WO 97/26243, thereby reducing the number of steps required.

### SUMMARY OF THE INVENTION

The present invention relates to a method of preparing a compound of the formula:

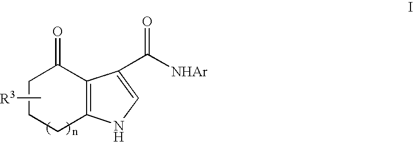

comprising reacting a compound of the formula:

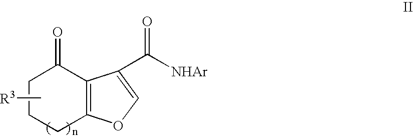

with an excess of ammonia source in a reaction inert solvent at an elevated temperature until reaction is complete;

wherein Ar is phenyl or heterocycle, said phenyl or heterocycle being substituted with —O—(CH₂)ₘ—NR¹R², —O(CH₂)ₜC(O)OR⁴, —CH(NR⁷R⁸)CH₃, —CH₂CH(NR⁵R⁶)CH₃, or OH, and said phenyl or heterocycle being optionally substituted with one or two groups selected from C₁-C₆ alkoxy, C₁-C₆ alkyl, C₂-C₆ alkenyl, C₁-C₆ perflouroalkyl, F, Cl, or Br, wherein:

R¹, R³, R⁴, R⁵ and R⁷ are independently selected from hydrogen and C₁-C₆ alkyl;

R², R⁶, and R⁸ are independently selected from nitrogen protecting groups;

m and l are integers independently selected from 1 to 6; and

n is an integer from 0 to 2.

In one embodiment, Ar is phenyl substituted with said one or two groups.

In another embodiment, the nitrogen protecting group is —C(O)C₁-C₆ alkoxy.

In another embodiment, the nitrogen protecting group is selected from the group consisting of benzyloxycarbonyl, fluorenyloxycarbonyl, acetyl, trifluoracetyl, chloroacetyl, benzoyl, t-butyloxycarbonyl, and benzyl.

In another embodiment, the compound of formula I is selected from the group consisting of

Methyl-(1-{4-[(4-oxo-4,5,6,7-tetrahydro-1H-indole-3-carbonyl)-amino]-phenyl}-ethyl)-carbamic acid tert-butyl ester;

[2-(2-Fluoro-4-[(4-oxo-4,5,6,7-tetrahydro-1H-indole-3-carbonyl)-amino]-phenoxy)-ethyl]-propyl-carbamic acid tert-butyl ester;

Butyl-(2-{5-[(4-oxo-4,5,6,7-tetrahydro-1H-indole-3-carbonyl)-amino]-pyridin-2-yloxy}-ethyl)-carbamic acid tert-butyl ester;

4-Oxo-4,5,6,7,8-hexahydro-cyclohepta[b]pyrrole-3-carboxylic acid (2-fluoro-4-hydroxy-phenyl)-amide;

(1-Methyl-2-{4-[(4-oxo-4,5,6,7-tetrahydro-1H-indole-3-carbonyl)-amino]-phenyl}-ethyl)-carbamic acid tert-butyl ester;

(2-{4-[(4-Oxo-4,5,6,7-tetrahydro-1H-indole-3-carbonyl)-amino]-phenoxy}-ethyl)-propyl-carbamic acid tert-butyl ester; and

{2-Fluoro-5-[(4-oxo-4,5,6,7-tetrahydro-1H-indole-3-carbonyl)-amino]-phenoxy}-acetic acid ethyl ester.

In a further embodiment, the compound of formula II is prepared by

(a) reacting a compound of the formula

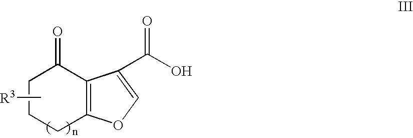

with an excess of an acid chloride or anhydride in a reaction inert solvent containing an excess of an acid acceptor until reaction is complete; and

(b) adding an equivalent amount of NH₂—Ar to the solution of step (a) and holding until reaction is complete.

In one embodiment, the acid chloride is ethylchloroformate.

In another embodiment, the method further comprises removing the nitrogen protecting group of formula I. For example, this can be accomplished by reacting the product of formula I with water in the presence of acid.

The invention also relates to a compound of the following formula:

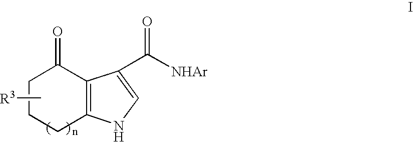

Preferably, the compound is selected from the group consisting of:

Methyl-(1-{4-[(4-oxo-4,5,6,7-tetrahydro-1H-indole-3-carbonyl)-amino]-phenyl}-ethyl)-carbamic acid tert-butyl ester;

[2-(2-Fluoro-4-[(4-oxo-4,5,6,7-tetrahydro-1H-indole-3-carbonyl)-amino]-phenoxy)-ethyl]-propyl-carbamic acid tert-butyl ester;

Butyl-(2{5-[(4-oxo-4,5,6,7-tetrahydro-1H-indole-3-carbonyl)-amino]-pyridin-2-yloxy}-ethyl)-carbamic acid tert-butyl ester;

4-Oxo-4,5,6,7,8-hexahydro-cyclohepta[b]pyrrole-3-carboxylic acid (2-fluoro-4-hydroxy-phenyl)-amide;

(1-Methyl-2-{4-[(4-oxo-4,5,6,7-tetrahydro-1H-indole-3-carbonyl)-amino]-phenyl}-ethyl)-carbamic acid tert-butyl ester;

(2-{4-[(4-Oxo-4,5,6,7-tetrahydro-1H-indole-3-carbonyl)-amino]-phenoxy}-ethyl)-propyl-carbamic acid tert-butyl ester; and

{2-Fluoro-5-[(4-oxo-4,5,6,7-tetrahydro-1H-indole-3-carbonyl)-amino]-phenoxy}-acetic acid ethyl ester.

The invention also relates to a compound of the following formula:

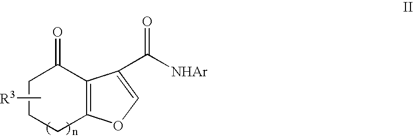

Preferably, the compound is selected from the group consisting of:

Methyl-(1-{4-[(4-oxo-4,5,6,7-tetrahydro-benzofuran-3-carbonyl)-amino]-phenyl}-ethyl)-carbamic acid tert-butyl ester;

[2-(2-Fluoro-4-[(4-oxo-4,5,6,7-tetrahydro-benzofuran-3-carbonyl)-amino]-phenoxy)-ethyl]-propyl-carbamic acid tert-butyl ester;

Butyl-(2-{5-[(4-oxo-4,5,6,7-tetrahydro-benzofuran-3-carbonyl)-amino]-pyridin-2-yloxy}-ethyl)-carbamic acid tert-butyl ester;

4-Oxo-4,5,6,7,8-hexahydro-cyclohepta[b]furan-3-carboxylic acid (2-fluoro-4-hydroxy-phenyl)-amide;

(1-Methyl-2-{4-[(4-oxo-4,5,6,7-tetrahydrobenzofuran-3-carbonyl)-amino]-phenyl}-ethyl)-carbamic acid tert-butyl ester;

(2-{4-[(4-Oxo-4,5,6,7-tetrahydrobenzofuran-3-carbonyl)-amino]-phenoxy}-ethyl)-propyl-carbamic acid tert-butyl ester; and

{2-Fluoro-5-[(4-oxo-4,5,6,7-tetrahydrobenzofuran-3-carbonyl)-amino]-phenoxy}-acetic acid ethyl ester.

### DETAILED DESCRIPTION OF THE INVENTION

All patents and patent publications referenced herein are hereby incorporated by reference in their entireties.

In one embodiment, the method of this invention is illustrated by the scheme shown below.

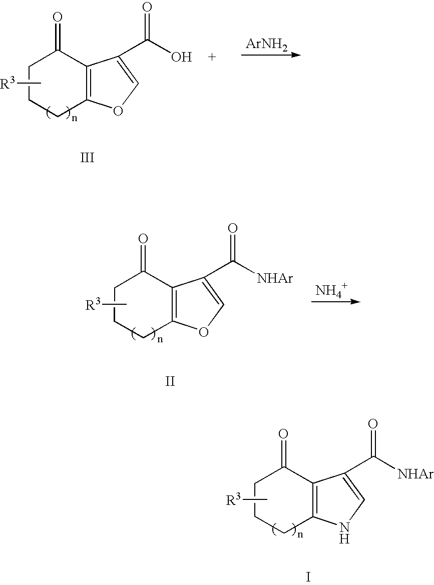

wherein Ar, R¹, R² and n are as defined above.

Compounds of formula III are readily prepared by reacting the appropriate 1,3-diketone with halo pyruvic acid ester, preferably ethylbromopyruvate as described in U.S. Pat. No. 5,484,944.

Compound II is prepared from compound III by converting the carboxylic acid group of compound I to the mixed acid anhydride and then to the carboxanilide by reaction of the acid anhydride with the selected aniline in the presence of base. The reaction is preferably carried out in a reaction inert solvent at a reduced temperature without isolation of the intermediate acid anhydride.

An acid chloride or anhydride may be used to form the mixed acid anhydride; ethylchloroformate is a preferred reagent.

The above-reaction is illustrated in general procedure A of Example 1 below.

Conversion of compound II to compound I is accomplished by reaction of compound II with an ammonium salt in a reaction inert solvent at an elevated temperature adequate to insure reaction in a reasonable period of time. A polar reaction inert solvent is suitable; n-methylpyrrolidine is preferred. Ammonium acetate is a convenient source of ammonium ion.

This procedure is illustrated in general procedure B of Example 1 below.

The reactions described herein employ N-protecting groups that are known in the art, including, for example, CBZ (benzyloxycarbonyl), FMOC (fluorenyloxycarbonyl), acetyl, trifluoracetyl, chloroacetyl, benzoyl, t-butyloxycarbonyl, and benzyl. Such protecting groups are described, for example, in Greene and Wuts, “Protective Groups in Organic Synthesis,” 2^{nd} Ed., chapter 7, 1991, John Wiley & Sons, New York.

Those skilled in this art will recognize that the starting materials may be varied and additional steps employed to produce compounds encompassed by the present invention, as demonstrated by the following examples. In some cases protection of certain reactive functionalities may be necessary to achieve some of the above transformations. In general the need for such protecting groups will be apparent to those skilled in the art of organic synthesis as well as the conditions necessary to attach and remove such groups.

The compounds formed by removal of a nitrogen protecting group in formula I may be administered orally, topically, parenterally, by inhalation or spray or rectally in dosage unit formulations containing conventional non-toxic pharmaceutically acceptable carriers, adjuvants and vehicles. The term parenteral includes subcutaneous injections, intravenous, intramuscular, intrasternal injection or infusion techniques. Pharmaceutical compositions containing compounds formed by deprotection of the compounds of formula I may be in a form suitable for oral use, for example, as tablets, troches, lozenges, aqueous or oily suspensions, dispersible powders or granules, emulsion, hard or soft capsules, or syrups or elixirs.

These compounds are highly selective agonists, antagonists or inverse agonists for GABAa brain receptors or prodrugs of agonists, antagonists or inverse agonists for GABAa brain receptors. Thus, these compounds are useful in the diagnosis and treatment of anxiety, sleep and seizure disorders, overdose with benzodiazepine drugs and for enhancement of memory. For example, these compounds can be used to treat overdoses of benzodiazepine-type drugs as they would competitively bind to the benzodiazepine receptor.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient.

The invention is illustrated further by the following examples which are not to be construed as limiting the invention in scope or spirit to the specific procedures described in them.

### EXAMPLE 1

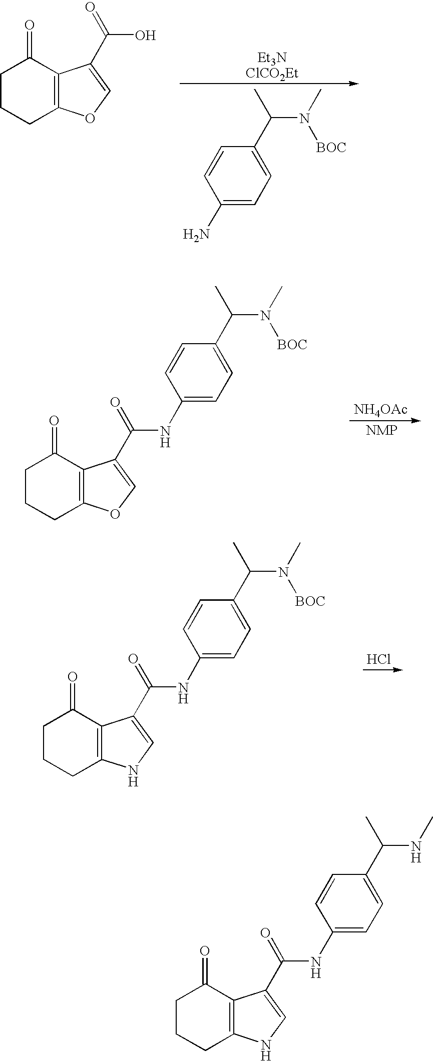

Methyl-(1-{4-[(4-oxo-4,5,6,7-tetrahydro-benzofuran-3-carbonyl)-amino]-phenyl}-ethyl)-carbamic acid tert-butyl ester

### General Procedure A:

A 0° C. solution of 4,5,6,7-tetrahydro-benzofuran-3-carboxylic acid (1.51 g, 8.36 mmol) in 20 mL dichloromethane was treated with triethylamine (1.50 mL, 10.9 mmol) and ethyl chloroformate (0.88 mL, 9.2 mmol). After 5 min, TLC analysis (3:1 hexane-EtOAc) showed complete conversion of the starting acid to a higher Rf spot. Methyl-[1-(4-aminophenyl)-ethyl]-10 carbamic acid tert-butyl ester was then added as a solution in 5 mL dichloromethane, rinsing with an additional 2 mL dichloromethane. The resulting solution was allowed to warm to ambient temperature overnight. The solution was diluted with dichloromethane and transferred to a separatory funnel, washed with two portions of water, one portion of brine, dried over MgSO₄, filtered, and concentrated to provide the product as an off-white solid (3.50 g, ca. 100% yield). Further purification, if required, can be achieved at this stage by silica gel chromatography or recrystallization from hexane-EtOAc:

¹H nmr (CDCl₃): 8.17 (s, 1H), 7.79 (d, 2H), 7.29 (d, 2H), 5.6-5.4 (br s, 1H), 3.01 (m, 2H), 2.72 (m, 2H), 2.60 (br s, 3H), 2.29 (m, 2H), 1.64 (m, 1H), 1.53 (m, 12H)

Methyl-(1-{4-[(4-oxo-4,5,6,7-tetrahydro-1H-indole-3-carbonyl)-amino]-phenyl}-ethyl)-carbamic acid tert-butyl ester

### General Procedure B:

A solution of methyl-(1-{4-[(4-oxo-4,5,6,7-tetrahydro-benzofuran-3-carbonyl)-amino]-phenyl}-ethyl)-carbamic acid tert-butyl ester: (1.75 g, 5.6 mmol) in N-methylpyrrolidinone (5 mL) was treated with NH₄OAc (1.64 g, 21.2 mmol) and placed in an 100° C. oil bath for 2 h, at which point TLC analysis (1:1 hexane-EtOAc) showed complete conversion to a lower Rf spot. After cooling to room temperature, the reaction mixture was diluted with EtOAc, washed with six portions of water, dried over MgSO₄, filtered, and concentrated to provide the desired product as a light brown solid (1.4 g, 4.5 mmol, 80% yield). Further purification, if required, can be achieved at this stage by silica gel chromatography or recrystallization from hexane-EtOAc:

C₂₃H₂₉N₃O₄ (m.w. 411.2)

MS (Cl): 410 (M−1)

4-Oxo-4,5,6,7-tetrahydro-1H-indole-3-carboxylic acid [4-(1-methylamino-ethyl)-phenyl]-amide

### General Procedure C:

A solution of methyl-(1-{4-[(4-oxo-4,5,6,7-tetrahydro-1H-indole-3-carbonyl)-amino]-phenyl}-ethyl)-carbamic acid tert-butyl ester (1.15 g, 3.7 mmol) in 40 mL of 3:1 MeOH-dichloromethane was treated with gaseous HCl by bubbling a stream of HCl (g) through the solution for 10 min. After 24 hours, the solution was concentrated to provide the HCl salt as a beige solid (1.05 g). This material was converted to the free base by dissolving in water, adding 1 N NaOH until the pH was >10, and collecting the resulting solids. After drying in a vacuum oven, the desired product was obtained as a brown solid (0.71 g). Further purification, if required, can be achieved at this stage by silica gel chromatography or recrystallization from dichloromethane-EtOAc:

m.p. 248-253° C.

C₁₈H₂₁N₃O₂ (m.w. 311.2)

MS (Cl): 310 (M−1)

### EXAMPLE 2

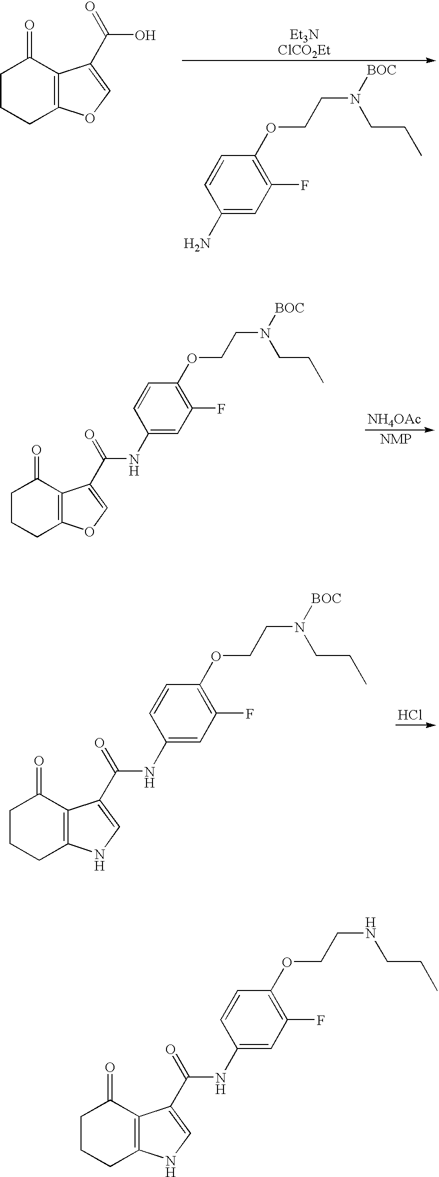

[2-(2-Fluoro-4-[(4-oxo-4,5,6,7-tetrahydro-benzofuran-3-carbonyl)-amino]-phenoxy)-ethyl]-propyl-carbamic acid tert-butyl ester

Starting with 4,5,6,7-tetrahydro-benzofuran-3-carboxylic acid and 2-(4-amino-2-fluoro-phenoxy)-ethyl-propyl-carbamic acid tert-butyl ester, General Procedure A provided the title compound as a white solid:

¹H nmr (CDCl₃): 11.7 (s, 1H), 8.10 (s, 1H), 7.72 (m, 1H), 7.37 (m, 1H), 6.92 (m, 1H0, 4.12 (m, 2H), 3.59 (m, 2H), 3.24 (m, 2H), 2.96 (m, 2H), 2.66 (m, 2H), 2.23 (m, 2H), 1.58 (m, 2H), 1.43 (s, 9H), 0.86 (t, 3H)

[2-(2-Fluoro-4-[(4-oxo-4,5,6,7-tetrahydro-1H-indole-3-carbonyl)-amino]-phenoxy)ethyl]-propyl-carbamic acid tert-butyl ester

Following General Procedure B from [2-(2-fluoro-4-[(4-oxo-4,5,6,7-tetrahydro-benzofuran-3-carbonyl)-amino]-phenoxy)-ethyl]-propyl-carbamic acid tert-butyl ester provided the title compound as a pale yellow oil:

¹H nmr (CDCl₃): 7.83 (m, 1H), 7.51 (s, 1H), 7.33 (m, 1H), 6.94 (m, 1H), 4.13 (m, 2H), 3.59 (m, 2H), 3.25 (m, 2H), 2.86 (m, 2H), 2.60 (m, 2H), 2.18 (m, 2H), 1.58 (m, 2H), 1.42 (s, 9H), 0.85 (t, 3H)

4-Oxo-4,5,6,7-tetrahydro-1H-indole-3-carboxylic acid [3-fluoro-4-(2-propylamino-ethoxy)-phenyl]-amide

Following General Procedure C from [2-(2-Fluoro-4-[(4-oxo-4,5,6,7-tetrahydro-1H-indole-3-carbonyl)-amino]-phenoxy)-ethyl]-propyl-carbamic acid tert-butyl ester provided the title compound.

### EXAMPLE 3

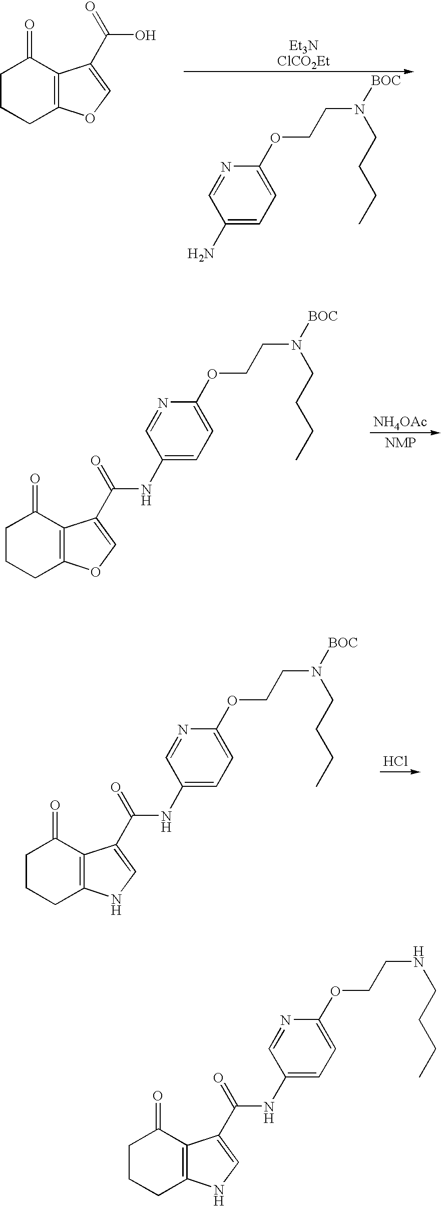

Butyl-(2-{5-[(4-oxo-4,5,6,7-tetrahydro-benzofuran-3-carbonyl)-amino]-pyridin-2-yloxy}-ethyl)-carbamic acid tert-butyl ester

Starting with 4,5,6,7-tetrahydro-benzofuran-3-carboxylic acid and [2-(5-aminopyridin-2-yloxy)-ethyl]-butyl-carbamic acid tert-butyl ester, General Procedure A provided the title compound as a white solid:

¹H nmr (CDCl₃): 8.48 (s, 1H), 8.12 (s, 1H), 8.08 (m, 1H), 6.72 (m, 1H), 4.39 (m, 2H), 3.54 (m, 2H), 3.24 (m, 2 h), 2.98 (m, 2H), 2.66 (m, 2H), 2.21 (m, 2H), 1.48 (m, 2H), 1.43 (s, 9H), 1.27 (M, 2H), 0.87 (t, 3H)

Butyl-(2-{5-[(4-oxo-4,5,6,7-tetrahydro-1H-indole-3-carbonyl)-amino]-pyridin-2-yloxy}-ethyl)-carbamic acid tert-butyl ester

Following General Procedure B from butyl-(2-{5-[(4-oxo-4,5,6,7-tetrahydro-benzofuran-3-carbonyl)-amino]-pyridin-2-yloxy}-ethyl)-carbamic acid tert-butyl ester provided the title compound as a pale yellow oil:

¹H nmr (CDCl₃): 12.23 (s, 1H), 9.10 (m, 1H), 8.70 (s, 1H), 8.06 (m, 1H), 7.56 (s, 1H), 6.89 (m, 1H), 4.51 (m, 2H), 3.29 (m, 2H), 2.92 (m, 2H), 2.81 (m, 2H), 2.58 (m, 2H), 2.03 (m, 2H), 1.80 (m, 2H), 1.32 (m, 2H), 0.83 (t, 3H)

C₂₅H₃₄N₄O₅ (m.w. 470)

MS (Cl): 469 (M−1)

4-Oxo-4,5,6,7-tetrahydro-1H-indole-3-carboxylic acid [6-(2-butylamino-ethoxy)-pyridin-3-yl]-amide

Following General Procedure C with butyl-(2-{5-[(4-oxo-4,5,6,7-tetrahydro-1H-indole-3-carbonyl)-amino]-pyridin-2-yloxy}-ethyl)-carbamic acid tert-butyl ester provided the desired compound as a gummy, brown solid.

### EXAMPLE 4

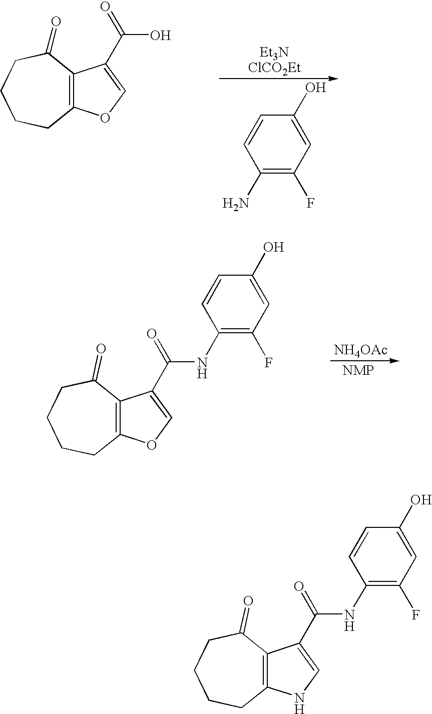

4-Oxo-4,5,6,7,8-hexahydro-cyclohepta[b]furan-3-carboxylic acid (2-fluoro-4-hydroxy-phenyl)-amide

Starting with 4-oxo-5,6,7,8-tetrahydro-4H-cyclohepta[b]furan-3-carboxylic acid and 2-fluoro-4-hydroxyaniline, General Procedure A provided the title compound as a white solid:

¹H nmr (d₆-DMSO): 11.38 (s, 1H), 9.79 (s, 1H), 8.23 (s, 1H), 7.88 (m, 1H), 6.60 (m, 2H), 3.09 (m, 2H), 2.77 (m, 2H), 1.82 (m, 4H)

4-Oxo-4,5,6,7,8-hexahydro-cyclohepta[b]pyrrole-3-carboxylic acid (2-fluoro-4-hydroxy-phenyl)-amide:

Following General Procedure B from 4-Oxo-4,5,6,7-tetrahydro-cyclohepta[b]furan-3-carboxylic acid-(2-fluoro-4-hydroxy-phenyl)-amide provided the title compound as a pale yellow oil:

¹H nmr (d₆-DMSO): 12.2 (s, 1H), 12.0 (br s, 1H), 9.61 (br s, 1H), 7.97 (m, 1H), 7.42 (s, 1H), 6.57 (m, 2H), 2.90 (m, 2H), 2.64 (m, 2H), 1.69 (m, 4H)

### EXAMPLE 5

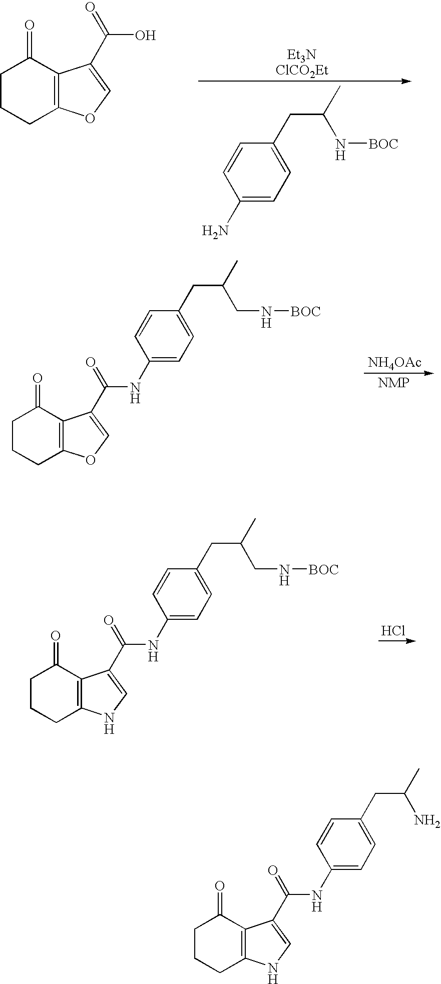

(1-M ethyl-2{(4-[(4-oxo-4,5,6,7-tetrahydrobenzofuran-3-carbonyl)-amino]-phenyl}-ethyl)-carbamic acid tert-butyl ester:

Starting with 4,5,6,7-tetrahydro-benzofuran-3-carboxylic acid and 1-methyl-2-(4-amino-phenyl)-ethyl-carbamic acid tert-butyl ester, General Procedure A provided the title compound as a white solid:

C₂₃H₂₈N₂O₅ (m.w. 412.2)

MS (Cl): 411 (M−1)

(1-Methyl-2{4-[(4-oxo-4,5,6,7-tetrahydro-1H-indole-3-carbonyl)-amino]-phenyl}-ethyl)-carbamic acid tert-butyl ester

Following General Procedure B from (1-methyl-2{4-[(4-oxo-4,5,6,7-tetrahydrobenzofuran-3-carbonyl)-amino]-phenyl}-ethyl)-carbamic acid tert-butyl ester provided the title compound as a pale yellow oil:

¹H nmr (CDCl₃): (several signals obscured by residual N-methylpyrrolidinone solvent) 11.82 (br s, 1H), 7.78 (d, 2H), 7.53 (s, 1H), 7.18 (d, 2H), 4.41 (br s, 1H), 3.85 (br s, 1H), 2.62 (m, 3H), 2.18 (m, 2H), 1.41 (s, 9H), 1.07 (d, 3H)

4-Oxo-4,5,6,7-tetrahydro-1H-indole-3-carboxylic acid [4-(2-amino-propyl)-phenyl]-amide

General Procedure C with (1-methyl-2{4-[(4-oxo-4,5,6,7-tetrahydro-1H-indole-3-carbonyl)-amino]-phenyl}-ethyl)-carbamic acid tert-butyl ester provided the title compound.

C₁₈H₂₁N₃O₂ (m.w. 311.2)

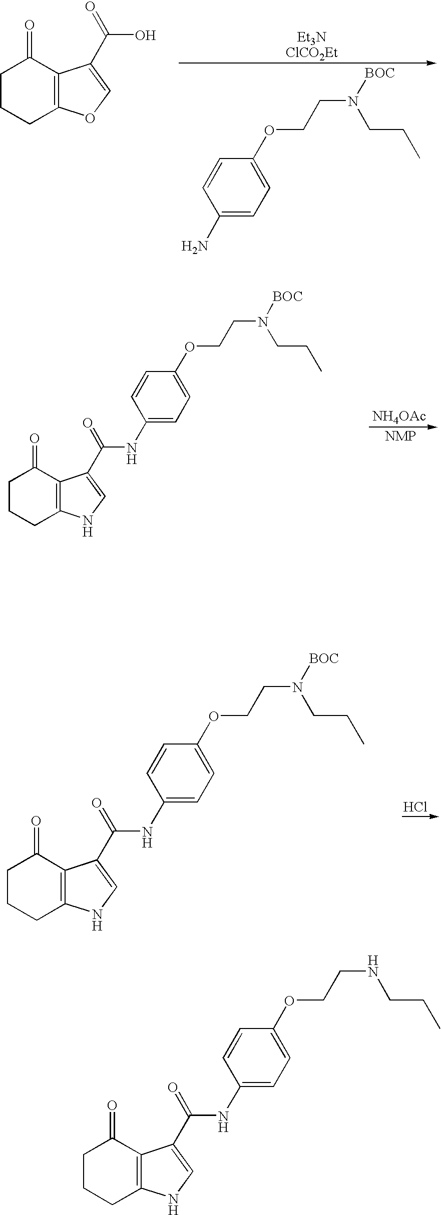

(2-{4-[(4-Oxo-4,5,6,7-tetrahydrobenzofuran-3-carbonyl)-amino]-phenoxy}-ethyl)-propyl-carbamic acid tert-butyl ester

Starting with 4,5,6,7-tetrahydro-benzofuran-3-carboxylic acid and [2-(4-aminophenoxy)-ethyl]-propyl-carbamic acid tert-butyl ester, General Procedure A provided the title compound as a white solid:

¹H nmr (CDCl₃): 11.62 (s, 1H), 8.12 (s, 1H), 7.69 (d, 2H), 6.82 (d, 2H), 4.03 (m, 2H), 3.54 (m, 2H), 3.22 (m, 2H), 2.98 (m, 2H), 2.65 (m, 2H), 2.23 (m, 2H), 1.53 (m, 2H), 1.45 (s, 9H), 0.83 (t, 3H)

(2-{4-[(4-Oxo-4,5,6,7-tetrahydro-1H-indole-3-carbonyl)-amino]-phenoxy}-ethyl)-propyl-carbamic acid tert-butyl ester

Following General Procedure B (2-{4-[(4-oxo-4,5,6,7-tetrahydrobenzofuran-3-carbonyl)-amino]-phenoxy}-ethyl)-propyl-carbamic acid tert-butyl ester provided the title compound as an off-white solid:

¹H nmr (CDCl₃): 11.39 (br s, 1H), 7.76 (d, 2H), 7.50 (s, 1H), 6.86 (d, 2H), 4.06 (m, 2H), 3.57 (m, 2H), 3.23 (m, 2H), 2.79 (m, 2H), 2.57 (m, 2H), 2.12 (m, 2H), 1.58 (m, 2H), 1.42 (w, 9H), 0.82 (t, 3H)

C₂₅H₃₃N₃O₅ (m.w. 455)

MS (C1): 454.3 (M−1, 100)

4-Oxo-4,5,6,7-tetrahydro-1H-indole-3-carboxylic acid [4-(2-propylamino-ethoxy)-phenyl]-amide

Starting with (2-{4-[(4-oxo-4,5,6,7-tetrahydro-1H-indole-3-carbonyl)-amino]-phenoxy}-ethyl)-propyl-carbamic acid tert-butyl ester, General Procedure C provided the target compound as an off-white solid.

¹H nmr (CD₃OD): 7.68 (d, 2H), 7.52 (s, 1H), 7.0 (d, 2H), 4.25 (m, 2H), 3.43 (m, 2H), 3.04 (m, 2H), 2.89 (m, 2H), 2.62 (m, 2H), 2.18 (m, 2H), 1.78 (m, 2H), 1.03 (t, 3H)

### EXAMPLE 7

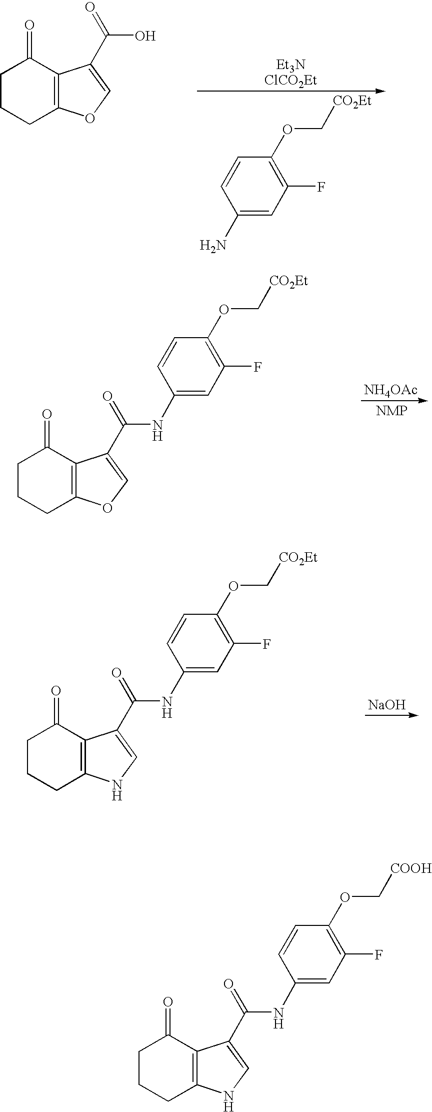

{2-Fluoro-4-[(4-oxo-4,5,6,7-tetrahydrobenzofuran-3-carbonyl)-amino]-phenoxy}-acetic acid ethyl ester

Starting with 4,5,6,7-tetrahydro-benzofuran-3-carboxylic acid and 2-fluoro-4-amino-phenoxyacetic acid ethyl ester, General Procedure A provided the title compound as a white solid:

¹H nmr (CDCl₃): 11.8 (br s, 1H), 8.11 (s, 1H), 7.79 (d, 1H), 7.38 (br d, 1H), 6.95 (t, 1H), 4.67 (s, 2H), 4.25 (q, 2H), 2.98 (m, 2H), 2.65 (m, 2H), 2.23 (m, 2H), 1.30 (t, 3H).

{2-Fluoro-4-[(4-oxo-4,5,6,7-tetrahydro-1H-indole-3-carbonyl)-amino]-phenoxy}-acetic acid ethyl ester

Following General Procedure B from {2-fluoro-4-[(4-oxo-4,5,6,7-tetrahydrobenzofuran-3-carbonyl)-amino}-phenoxy]acetic acid ethyl ester provided the title compound as an off-white solid:

¹H nmr (CDCl₃): 10.65 (br s, 1H), 7.91 (d, 1H), 7.57 (s, 1H), 7.35 (d, 1H), 6.97 (t, 1H), 4.67 (s, 2H), 4.25 (q, 2H), 2.87 (m, 2H), 2.61 (m, 2H), 2.19 (m, 2H), 1.28 (t, 3H).

{2-Fluoro-4-[(4-oxo-4,5,6,7-tetrahydro-1H-indole-3-carbonyl)-amino]-phenoxy}-acetic acid

{2-Fluoro-4-[(4-oxo-4,5,6,7-tetrahydro-1H-indole-3-carbonyl)-amino]-phenoxy}-acetic acid ethyl ester (9.35 g) dissolved in 40 mL EtOH was treated with 160 mL 1 N NaOH and warmed to reflux for 90 min. After cooling to room temperature, the solution was washed with EtOAc, cooled in an ice bath, and acidified to pH 2 with 3 N HCl. The resulting solid was filtered, washed with water and EtOAc, then dried in vacuo to provide the desired product as a light brown solid (7.88 g).

¹H nmr (d₆-DMSO): 12.1 (br s, 1H), 7.80 (br d, 1H), 7.57 (br s, 1H), 7.22 (br d, 1H), 7.08 (br t, 1H), 4.66 (s, 2H), 2.83 (m, 2H), 2.57 (m, 2H), 2.04 (m, 2H).

MS (Cl): 347 (M+1)

C₁₇H₁₅FN₂O₅ (m.w. 346.3)

We claim:

1. A method of preparing a compound of the formula: 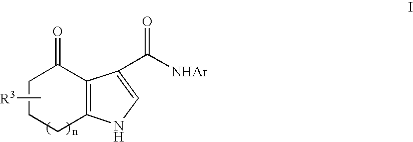
comprising reacting a compound of the formula: 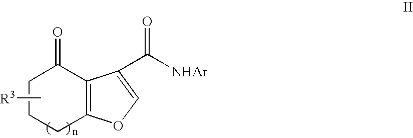
with an excess of ammonia source in a reaction inert solvent at an elevated temperature until reaction is complete;
wherein Ar is phenyl or heterocycle, said phenyl or heterocycle being substituted with —O—(CH₂)ₘ—NR¹R², —O(CH₂)ₜC(O)OR⁴, —CH(NR⁷R⁸)CH₃, —CH₂CH(NR⁵R⁶)CH₃, or OH, and said phenyl or heterocycle being optionally substituted with one or two groups selected from C₁-C₆ alkoxy, C₁-C₆ alkyl, C₂-C₆ alkenyl, C₁-C₆ perflouroalkyl, F, Cl, and Br, wherein:
R¹, R³, R⁴, R⁵ and R⁷ are independently selected from hydrogen and C₁-C₆ alkyl;
R², R⁶, and R⁸ are independently selected from nitrogen protecting groups;
m and l are integers independently selected from 1 to 6; and
n is an integer from 0 to 2.

2. The method of claim 1 wherein Ar is phenyl substituted with said one or two groups.

3. The method of claim 1 wherein said nitrogen protecting group is —C(O)C₁-C₆ alkoxy.

4. The method of claim 1 wherein said nitrogen protecting group is benzyloxycarbonyl, fluorenyloxycarbonyl, acetyl, trifluoracetyl, chloroacetyl, benzoyl, t-butyloxycarbonyl, or benzyl.

5. The method of claim 1 wherein said compound of formula I is selected from the group consisting of
Methyl-(1-{4-[(4-oxo-4,5,6,7-tetrahydro-1H-indole-3-carbonyl)-amino]-phenyl}-ethyl)-carbamic acid tert-butyl ester;
[2-(2-Fluoro-4-[(4-oxo-4,5,6,7-tetrahydro-1H-indole-3-carbonyl)-amino]-phenoxy)-ethyl]-propyl-carbamic acid tert-butyl ester;
Butyl-(2-{5-[(4-oxo-4,5,6,7-tetrahydro-1H-indole-3-carbonyl)-amino]-pyridin-2-yloxy}-ethyl)-carbamic acid tert-butyl ester;
4-Oxo-4,5,6,7,8-hexahydro-cyclohepta[b]pyrrole-3-carboxylic acid (2-fluoro-4-hydroxy-phenyl)-amide;
(1-Methyl-2-{4-[(4-oxo-4,5,6,7-tetrahydro-1H-indole-3-carbonyl)-amino]-phenyl}-ethyl)-carbamic acid tert-butyl ester;
(2-{4-[(4-Oxo-4,5,6,7-tetrahydro-1H-indole-3-carbonyl)-amino]-phenoxy}-ethyl)-propyl-carbamic acid tert-butyl ester; and
{2-Fluoro-5-[(4-oxo-4,5,6,7-tetrahydro-1H-indole-3-carbonyl)-amino]-phenoxy}-acetic acid ethyl ester.

6. A method according to claim 1 further wherein said compound of formula II is prepared by
(a) reacting a compound of the formula 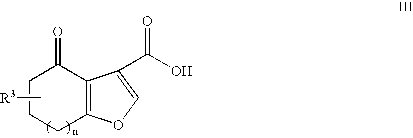
with an excess of an acid chloride or anhydride in a reaction inert solvent containing an excess of an acid acceptor until reaction is complete; and
(b) adding an equivalent amount of NH₂—Ar to the solution of step (a) and holding until reaction is complete.

7. The method of claim 6 wherein said acid chloride is ethylchloroformate.

8. The method according to claim 1 which further comprises removing said nitrogen protecting group.

9. The method according to claim 5 which further comprises removing said nitrogen protecting group.

10. A compound of the following formula: 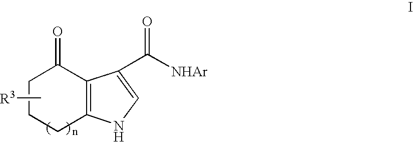
wherein Ar is phenyl or heterocycle, said phenyl or heterocycle being substituted with —O—(CH₂)ₘ—NR¹R², —O(CH₂)ₜC(O)OR⁴—CH(NR⁷R⁸)CH₃, —CH₂CH(NR⁵R⁶)CH₃, or OH, and said phenyl or heterocycle being optionally substituted with one or two groups selected from C₁-C₆ alkoxy, C₁-C₆ alkyl, C₂-C₆ alkenyl, C₁-C₆ perflouroalkyl, F, Cl, and Br, wherein:
R¹, R³, R⁴, R⁵ and R⁷ are independently selected from hydrogen and C₁-C₆ alkyl;
R², R⁶, and R⁸ are independently selected from nitrogen protecting groups;
m and l are integers independently selected from 1 to 6; and
n is an integer from 0 to 2.

11. A compound of claim 10 selected from the group consisting of:
Methyl-(1-{4-[(4-oxo-4,5,6,7-tetrahydro-1H-indole-3-carbonyl)-amino]-phenyl}-ethyl)-carbamic acid tert-butyl ester;
[2-(2-Fluoro-4-[(4-oxo-4,5,6,7-tetrahydro-1H-indole-3-carbonyl)-amino]-phenoxy)-ethyl]-propyl-carbamic acid tert-butyl ester;
Butyl-(2-{5-[(4-oxo-4, 5,6,7-tetrahydro-1H-indole-3-carbonyl)-amino]-pyrid in-2-yloxy}-ethyl)-carbamic acid tert-butyl ester;
4-Oxo-4,5,6,7,8-hexahydro-cyclohepta[b]pyrrole-3-carboxylic acid (2-fluoro-4-hydroxy-phenyl)-amide;
(1-Methyl-2-{4-[(4-oxo-4,5,6,7-tetrahydro-1H-indole-3-carbonyl)-amino]-phenyl}-ethyl)-carbamic acid tert-butyl ester;
(2-{4-[(4-Oxo-4,5,6,7-tetrahydro-1H-indole-3-carbonyl)-amino]-phenoxy}-ethyl)-propyl-carbamic acid tert-butyl ester; and
{2-Fluoro-5-[(4-oxo-4,5,6,7-tetrahydro-1H-indole-3-carbonyl)-amino]-phenoxy}-acetic acid ethyl ester.

12. A compound of the following formula: 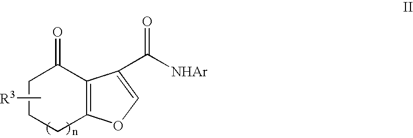
wherein Ar is phenyl or heterocycle, said phenyl or heterocycle being substituted with —O—(CH₂)ₘ—NR¹R², —O(CH₂)ₜC(O)OR⁴, —CH(NR⁷R⁸)CH₃, —CH₂CH(NR⁵R⁶)CH₃, or OH, and said phenyl or heterocycle being optionally substituted with one or two groups selected from C₁-C₆ alkoxy, C₁-C₆ alkyl, C₂-C₆ alkenyl, C₁-C₆ perflouroalkyl, F, Cl, and Br, wherein:
R¹, R³, R⁴, R⁵ and R⁷ are independently selected from hydrogen and C₁-C₆ alkyl;
R², R⁶, and R⁸ are independently selected from nitrogen protecting groups;
m and l are integers independently selected from 1 to 6; and
n is an integer from 0 to 2.

13. The compound of claim 12 selected from the group consisting of:
Methyl-(1-{4-[(4-oxo-4,5,6,7-tetrahydro-benzofuran-3-carbonyl)-amino]-phenyl}-ethyl)-carbamic acid tert-butyl ester;
[2-(2-Fluoro-4-[(4-oxo-4,5,6,7-tetrahydro-benzofuran-3-carbonyl)-amino]-phenoxy)-ethyl]-propyl-carbamic acid tert-butyl ester;
Butyl-(2-{5-[(4-oxo-4,5,6,7-tetrahydro-benzofuran-3-carbonyl)-amino]-pyrid in-2-yloxy}-ethyl)-carbamic acid tert-butyl ester;
4-Oxo-4,5,6,7,8-hexahydro-cyclohepta[b]furan-3-carboxylic acid (2-fluoro-4-hydroxy-phenyl)-amide;
(1-Methyl-2-{4-[(4-oxo-4,5,6,7-tetrahydrobenzofuran-3-carbonyl)-amino]-phenyl}-ethyl)-carbamic acid tert-butyl ester;
(2-{4-[(4-Oxo-4,5,6,7-tetrahydrobenzofuran-3-carbonyl)-amino]-phenoxy}-ethyl)-propyl-carbamic acid tert-butyl ester; and
{2-Fluoro-5-[(4-oxo-4,5,6,7-tetrahydrobenzofuran-3-carbonyl)-amino]-phenoxy} acetic acid ethyl ester.
